(19) United States
(12) Patent Application Publication
(10) Pub. No.: US 2012/0156092 A1
(43) Pub. Date: Jun. 21, 2012

(54) STERILIZATION COAT

(75) Inventor: Jan Frederik Suijver, Eindhoven (NL)

(73) Assignee: KONINKLIJKE PHILIPS ELECTRONICS N.V., EINDHOVEN (NL)

(21) Appl. No.: 13/389,284

(22) PCT Filed: Sep. 2, 2010
(86) PCT No.: PCT/IB2010/053947

§ 371 Date: Feb. 7, 2012

(30) Foreign Application Priority Data
Sep. 8, 2009 (EP) 09169733.4

(51) Int. Cl. A61L 2/04; A61L 2/28

(52) U.S. Cl. 422/22; 422/307; 422/119

(57) ABSTRACT

The invention relates to a sterilization coat (1) for sterilizing a device (2) like a medical detection device, wherein the sterilization coat (1) is adapted to be arranged on the device (2) and comprises a heating layer for sterilizing the device (2) by heating. If the sterilization coat (1) has been arranged on the device, several sterilization procedures can be performed by heating the sterilization coat (1) using the heating layer. Thus, it is not necessary to arrange sterile plastic drapes around the device (2) each time a sterile device is needed, for example, before each medical interventional procedure. If a sterilization of the device (2) is needed, the heating layer heats the device (2) for sterilizing. This simplifies the procedure for sterilizing the device (2).

### FIELD OF THE INVENTION

The invention relates to a sterilization coat for being arranged on a device for sterilizing the device, a sterilization apparatus comprising the sterilization coat for sterilizing the device, a device comprising the sterilization coat, and a sterilization method for sterilizing a device.

### BACKGROUND OF THE INVENTION

In many medical interventional procedures large equipment is used such as a radiation shield or an X-ray flat detector device. Due to the need for sterilization in a medical environment at least parts of this equipment are covered in sterile plastic drapes. Before the onset of a medical interventional procedure it can take up to half an hour to carefully apply the sterile plastic drapes to the medical equipment like the radiation shield or the X-ray flat detector device.

### SUMMARY OF THE INVENTION

It is an object of the present invention to simplify the sterilization of the equipment.

In a first aspect of the present invention a sterilization coat for sterilizing a device is presented, wherein the sterilization coat is adapted to be arranged on the device and comprises a heating layer for sterilizing the device by heating.

If the sterilization coat has been arranged on the device, several sterilization procedures can be performed by heating the sterilization coat using the heating layer. Thus, it is not necessary to arrange sterile plastic drapes around the device each time a sterile device is needed, for example, before each medical interventional procedure. If a sterilization of the device is needed, the heating layer heats the device for sterilizing. This simplifies the procedure for sterilizing the device.

The sterilization coat comprises preferentially a layer structure which is applied to an outer surface of the device. The sterilization coat is preferentially attachable to the device by an attachment means like an adhesive or clamping means.

The sterilization coat is preferentially adapted to heat to a temperature above 120° C., further preferred between 120° C. to 150° C. and even further preferred between 130° C. and 140° C. The sterilization coat is preferentially adapted to heat for at least one minute, further preferred for at least five minutes, and even further preferred for at least ten minutes.

The heating layer can be adapted to heat in different ways, for example, electrically, inductively, or by absorbing radiation, i.e. optically.

The heating layer comprises heating material arranged in a layer. The heating material is a material which can generate heat like metal, in particular, a metallic layer or a plurality of electrical wires arranged in the heating layer, for generating heat electrically or inductively.

It is preferred that the sterilization coat is adapted to be arranged on a detection device for detecting radiation, wherein the sterilization coat is transparent to the radiation.

The sterilization coat is preferentially substantially transparent to the radiation, i.e., in particular the sterilization coat has preferentially a transmission to the radiation detected by the detection device being larger than 75%, further preferred larger than 90%, and even more preferred larger than 95%.

The sterilization coat is preferentially adapted such that it can be used on a detection device without detrimentally effecting the operation of the detection device. In contrast, the above mentioned plastic drapes of the prior art induce image artifacts, if the detected radiation is used for generating an image. These image artifacts can hinder, for example, optical registration procedures which are based on the generated image.

The sterilization coat can, for example, be adapted for sterilizing an X-ray detector. In this case, the sterilization coat is preferentially transparent to X-rays and the heating layer is preferentially a layer of aluminum having a thickness of, for example, 150 nm. If in addition or alternatively the sterilization coat is adapted to sterilize a detection device for detecting optical radiation like radiation in the visible or infrared range, the heating material is preferentially an ITO layer. An ITO layer is optically transparent, electrically conductive and colorless in thin layers and comprises a solid solution of indium (III) oxide (In2O3) and tin(IV) oxide (SnO2), typically 90% In2O3, 10% SnO2 by weight.

It is further preferred that the heating layer has a first side and a second side being opposite to the first side, wherein the sterilization coat comprises a thermally isolating layer being arranged at the first side.

The first side of the heating layer is preferentially directed towards the device, if the sterilization coat is arranged on the device. The second side of the heating layer is preferentially directed towards the outer surface of the sterilization coat, if the sterilization coat is arranged on the device. The arrangement of the thermally isolating layer at the first side of the heating layer reduces the probability that the device is adversely affected by the sterilization heat. In an embodiment, the thermally isolating layer forms a surface of the sterilization coat, which is preferentially arranged on the device, if the sterilization coat has been applied to the device. In another embodiment, the thermally isolating layer can also be located between this surface and the heating layer.

It is further preferred that the thermally isolating layer is electrically isolating. This reduces the probability that the device is adversely affected by an electrical current which may flow through the heating layer for heating the sterilization coat.

The thermally isolating layer comprises preferentially at least one of polymerized plastics and glass. Preferred polymerized plastics are polystyrene, polyethylene terephthalate, polypropylene, et cetera. The sterilization coat preferentially comprises an electrically isolating layer that is thermally conductive and arranged at the second side of the heating layer. As already mentioned above, the second side of the heating layer is preferentially directed towards the outer surface of the sterilization coat, if the sterilization coat is arranged on the device. The electrically isolating layer that is thermally conductive can be arranged at this outer surface of the sterilization coat or at another position between this outer surface and the heating layer.

Since the electrically isolating layer is thermally conductive, the heat generated in the heating layer can be transferred through the electrically isolating layer to the outside of the device for sterilizing the device. Moreover, the electrical isolation improves the safety of the sterilization coat. The electrically isolating layer comprises preferentially at least one of parylene and glass. The electrically isolating layer has preferentially a thickness between 1 and 20 μm, further preferred between 5 and 15 μm and even further preferred of 10 μm.

The sterilization coat preferentially comprises a layer structure, wherein the heating layer is arranged on the thermally isolating layer and wherein the electrically isolating layer that is thermally conductive is arranged on the heating layer. Thus, the sterilization coat can form a sandwich structure, wherein the heating layer is sandwiched between the thermally isolating layer and the electrically isolating layer that is thermally conductive.

If the sterilization coat is adapted for being applied to a detection device, all layers of the layer structure are preferentially transparent to the kind of radiation detected by the detection device at least at the radiation sensitive areas of the detection device. Preferentially, the entire sterilization coat is transparent to the radiation detected by the detection device, i.e. the sterilization coat preferentially does not comprise unnecessary structures with regions being transparent and regions being not transparent, thereby simplifying manufacturing of the sterilization coat.

Thus, the sterilization coat is preferentially a single type of sterilization coat that is transparent to all kinds of radiation which may be detected by the detection device. For example, the sterilization coat can be transparent for both, X-rays and optical radiation, at each location of the sterilization coat. In this case, the heating layer is preferentially an ITO layer.

In an embodiment, the sterilization coat comprises a first region being transparent to a first kind of radiation and a second region being transparent to a second kind of radiation. This allows using the sterilization coat for sterilizing a device being adapted for detecting two kinds of radiations, for example, X-rays and optical radiation, in particular, in the infrared wavelength range. In an embodiment, the sterilization coat is adapted to be arranged on an X-ray flat detector plate that includes optical cameras, wherein the sterilization coat is adapted such that it can be arranged on the X-ray flat detector plate with the cameras such that the first region being transparent to X-rays covers the sensitive area of the X-ray flat detector plate and the second region being transparent to another kind of radiation detectable by the optical cameras covers the optical cameras. The sterilization coat can comprise one or several first regions and one or several second regions, which preferentially correspond to respective radiation sensitive areas of the detection device.

It is further preferred that the heating layer comprises a first heating material being transparent to the first kind of radiation in the first region and a second heating material being transparent to the second kind of radiation in the second region, wherein the first heating material and the second heating material are electrically connected to each other. This allows applying voltage to the first heating material and the second heating material simultaneously, i.e. the current can flow between the first heating material and the second heating material. This simplifies applying voltage to the heating material, if the sterilization coat is adapted to be heated electrically. Advantageously, the specific resistances of the two heating materials are substantially similar, allowing for a uniform temperature distribution across both heating materials within the heating layer.

In the first region the heating layer preferentially comprises aluminum and in the second region the heating layer preferentially comprises ITO.

It is preferred that in the first region the thermally isolating layer is transparent to the first kind of radiation and in the second region the thermally isolating layer is transparent to the second kind of radiation.

It is further preferred that in the first region the thermally isolating layer comprises at least one of polymerized plastics and glass and in the second region the thermally isolating layer comprises at least one of glass and quartz.

It is further preferred that the sterilization coat comprises an indicator for indicating if the heating material has sterilized the device. The indicator is preferentially located outside of the first region and the second region. The indicator is preferentially adapted to indicate when the heating layer has heated the sterilization coat for sterilizing the device.

The indicator preferentially comprises a thermochromic material. The thermochromic material changes its appearance, in particular, color, with temperature. If a sterilization procedure has been performed, the thermochromic material shows an appearance indicating an increased temperature. After some time the thermochromic material has cooled down to, for example, room temperature and the thermochromic material indicates the cooled down temperature by its appearance. This can indicate that a next sterilization procedure should be performed before using the device again.

The outer electrically isolating layer covers preferentially the entire heating layer.

In a further aspect of the present invention a sterilization apparatus for sterilizing a device by heating is presented, wherein the sterilization apparatus comprises:

a sterilization coat as defined in claim 1,

a control unit for controlling the heating by the heating layer.

It is preferred that the control unit and the heating layer are adapted to generate heat electrically for sterilization. In other embodiments, the control unit and the heating layer can be adapted to inductively or optically heat the heating layer.

It is further preferred that the sterilization apparatus comprises an indicator for indicating if the heating layer has been heated for sterilizing the device.

The indicator comprises, for example, a display and a storing unit, wherein in the storing unit the time of the last successful sterilization procedure is stored, which can be displayed on the display. The storing unit is preferentially connected to the control unit such that the control unit stores the time of sterilization, after a sterilization procedure has been performed.

In a further aspect of the present invention a device comprising the sterilization coat for sterilizing the device by heating is presented.

Preferentially, the device is a detection device for detecting radiation.

It is further preferred that the detection device comprises a first detector for detecting a first kind of radiation and a second detector for detecting a second kind of radiation. The first detector is preferentially adapted to detect X-rays and the second detector is preferentially adapted to detect optical radiation.

It is further preferred that the sterilization coat comprises a first region being transparent to the first kind of radiation and a second region being transparent to the second kind of radiation, wherein the first region is arranged on the first detector and the second region is arranged on the second detector.

In a further aspect of the present invention a sterilization method for sterilizing a device is presented, wherein a sterilization coat comprising a heating layer is arranged on the device and wherein the heating layer heats for sterilizing the device.

It shall be understood that the sterilization coat of claim 1, the sterilization apparatus of claim 9, the device of claim 11 and the sterilization method of claim 15 have similar and/or identical preferred embodiments as defined in the dependent claims.

It shall be understood that a preferred embodiment of the invention can also be any combination of the dependent claims with the respective independent claim. These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily a device with a sterilization apparatus for sterilizing the device, wherein the device is covered by a sterilization coat,

FIG. 2 shows schematically and exemplarily a layer structure of the sterilization coat,

FIG. 3 shows schematically and exemplarily another embodiment of the sterilization coat, and

FIG. 4 shows a flowchart exemplarily illustrating an embodiment of a sterilization method for sterilizing a device.

### DETAILED DESCRIPTION OF EMBODIMENTS

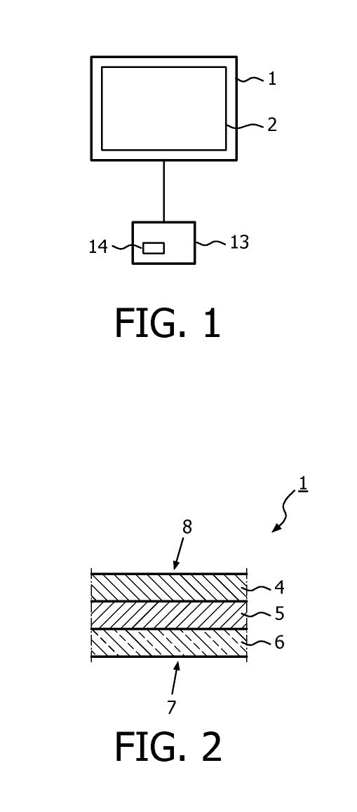

FIG. 1 shows schematically and exemplarily a device 2 enclosed by a sterilization coat 1. The sterilization coat 1 comprises a heating layer for sterilizing the device by heating. The sterilization coat 1 is attached to the device 2 by an adhesive.

The adhesive is preferentially a chemical adhesive like a cyanoacrylate glue or a two-component glue. These adhesives can have the advantage that they are strong while only requiring a very thin layer. These adhesives can therefore be substantially transparent for X-rays or optical radiation. Alternatively, a silicone-paste type of adhesive can be used. Such an adhesive can fulfill two functions, attaching the sterilization coat to the device and thermally isolating the device with respect to the heat generated by the heating layer. The silicone-paste type of adhesive can therefore be used as a thermally isolating layer,

The sterilization coat 1 is adapted to heat to a temperature between 120° C. and 140° C. for at least ten minutes for sterilizing the device 2. The heating layer is adapted to heat electrically. In other embodiments, the heating layer can be adapted to heat in another way, for example, inductively or optically.

The sterilization coat 1 comprises a layer structure which is schematically and exemplarily shown in FIG. 2.

The sterilization coat 1 comprises a first side 7 for being arranged on the device 2 and a second side 8 being opposite to the first side 7, wherein the second side 8 is formed by an outer electrically isolating layer 4 that is thermally conductive. In this embodiment, the outer electrically isolating layer 4 is a parylene layer. In other embodiments, the outer electrically isolating layer 4 can be another layer, for example, a glass layer. The outer electrically isolating layer 4 has a thickness of 10 μm. In other embodiments, the outer electrically isolating layer 4 can have another thickness, for example, a thickness between 1 and 20 μm, in particular, between 5 and 15 μm. The thickness can also be larger than 20 μm.

The first side 7 is formed by a thermally isolating layer 6. Also the thermally isolating layer 6 is electrically isolating. The thermally isolating layer 6 is made of a layer of polymerized plastics, preferentially, of polystyrene and/or polyethylene terephthalate and/or polypropylene. In other embodiments, the thermally isolating layer 6 can be made of another thermally isolating material like glass.

The heatable material is arranged in a heating layer 5. Thus, the sterilization coat 1 has a layer structure, wherein the heating layer 5 comprising the heatable material is arranged on the thermally isolating layer 6 forming the first side for being arranged on the device 2 and wherein the electrically isolating layer 4 forming the second side 7, i.e. forming an outer surface of the sterilization coat, if the sterilization coat is applied to the device 2, is arranged on the heating layer 5. The sterilization coat 1 has therefore a sandwich structure, wherein the heating layer 5 is sandwiched between the thermally isolating layer 6 for being arranged on the device 2 and the electrically isolating layer 4 that is thermally conductive forming the outer surface if the sterilization coat is applied to the device 2. The device 2 is a detection device for detecting radiation, wherein the sterilization coat 1 is transparent to the radiation. The transparency to the radiation allows the sterilization coat 1 to be used on the detection device 2 with only slightly or without a detrimentally effecting the operation of the detection device 2.

In this embodiment, the detection device 2 is an X-ray detector and the sterilization coat 1 is transparent to X-rays. The heating layer 5 is a layer of aluminum having a thickness of 150 nm. In other embodiments, the heating layer can, for example, be made of another metal being transparent to X-rays and/or the heating layer can have another thickness. Moreover, in another embodiment, the heating layer is transparent to optical radiation like radiation in the visible range. In this case, the heating layer is preferentially an ITO layer.

The electrically isolating layer 4 that is thermally conductive, the heating layer 5 and the thermally isolating layer 6 are transparent to the kind of radiation detected by the detection device 2, i.e. in this embodiment transparent to X-rays, at least at the radiation sensitive areas of the detection device 2. However, in this embodiment the entire sterilization coat 1 is transparent to X-rays, i.e. the sterilization coat 1 does not comprise a structure with areas being transparent and areas being not transparent.

The sterilization coat is preferentially applied to surfaces of the device, which have to be sterilized. Thus, it is not in any case necessary to apply the sterilization coat to all outer surfaces of the device.

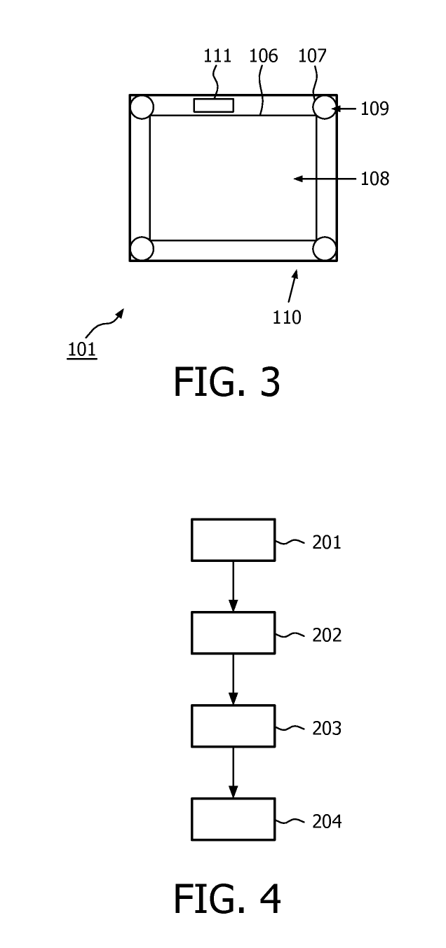

FIG. 3 shows schematically and exemplarily a top view on a sensitive area of a detection device, wherein the detection device is covered by a sterilization coat 101. The sterilization coat 101 comprises a first region 108 being transparent to a first kind of radiation and second regions 109 being transparent to a second kind of radiation. This allows using the sterilization coat 101 for sterilizing a device being adapted for detecting two kinds of radiation. In this embodiment the device covered by the sterilization coat 101 is a detection device for detecting X-rays and optical radiation. The detection device comprises an X-ray flat detector plate covered by the sterilization coat 101. The sterilization coat 101 can also cover further areas, in particular, further surfaces, of the detection device. The X-ray flat detector plate comprises a first sensitive area being sensitive for X-rays and being covered by the first region 108. This first sensitive area forms a first detector 106. The X-ray flat detector plate further comprises several, e.g. in FIG. 3 four, second sensitive areas being sensitive for optical radiation and being covered by the second regions 109 of the sterilization coat 101. Such second sensitive area forms a second detector 107, e.g. a camera capturing visual light. In FIG. 3, the first region 108 is an inner region, in particular, an inner rectangular region, surrounded by a rectangular frame 110. The rectangular frame 110 comprises the four second regions 109 at its four corners.

The sterilization coat 101 comprises a layer structure with at least an outer electrically isolating layer, an inner thermally isolating layer facing the detection device and a heating layer between the outer electrically isolating layer and the inner thermally isolating layer as schematically and exemplarily shown in FIG. 2. The layer structure of the sterilization coat 101 comprises different materials in the first region 108 and in the second regions 109. In the first region the thermally isolating layer comprises at least one of polymerized plastics and glass and in the second regions the thermally isolating layer comprises at least one of glass and quartz. Moreover, in the first region the heatable layer comprises a metal like aluminum and in the second regions the heatable layer comprises preferentially ITO. The outer electrically isolating layer is preferentially the same for the first region, the second region, in particular, the outer electrically isolating layer is preferentially the same for the entire sterilization coat 101. Preferentially, the electrically isolating layer covers at least the entire X-ray flat detector plate and preferentially further surfaces of the detection device, in particular, further surfaces which could come into contact with persons like a physician or a patient during an interventional procedure. In an embodiment, all outer surfaces of the detection device are covered by the sterilization coat 101. The outer electrically isolating layer is thermally conductive and, for example, a parylene layer and/or a thin glass layer.

The glass layer has preferentially a thickness between 0.01 and 5 mm, further preferred between 0.1 and 1 mm and even further preferred between 0.25 and 0.5 mm.

In another embodiment, the outer electrically isolating layer can be structured, in particular, the electrically isolating layer can comprise a parylene layer in the first region 108 and a glass layer in the second regions 109. The remaining part of the outer electrically isolating layer can comprise parylene, glass or both.

The parylene layer has preferentially a thickness of 10 μm-100 μm, and is preferentially a parylene-C or a parylene-F variant.

The structure of the sterilization coat 101 allows sterilizing the detection device very fast without detrimental effect to the optical and/or X-ray images detected by the detection device.

The heating layer in the first region 108 is in electrical contact with the heating layer in the second regions 109. This electrical contact is preferentially made at the periphery of the optical cameras, i.e. at the periphery of the second regions 109, by using an electrical contact means like silver paint. This allows an electrical current to flow between the heating layer in the first region 108 which can be regarded as a first heatable material and the heating layer in the second regions 109 which can be regarded as second heatable material. This simplifies applying voltage to the heatable material for heating the sterilization coat 101 electrically.

The sterilization coat 101 further comprises an indicator 111 located outside of the first region 108 and the second regions 109. The indicator 111 is adapted to indicate when the heating layer has heated for sterilizing the device. In particular, the indicator is adapted to indicate when the heating layer has heated the last time to a sterilization temperature above, for example, 140° C.

The outer electrically isolating layer that is thermally conductive forms preferentially the entire outer surface of the sterilization coat 101. If this layer is made of polymerized plastics, the polymerized plastics of the electrically and thermally isolating layer between the heating layer and the detection device are preferentially made of polystyrene or polyethylene terephthalate or polypropylene.

Referring again to FIG. 1, a control unit 13 is connected to the heating layer 5 of the sterilization coat 1. The control unit 13 comprises a voltage source for applying voltage to the heating layer 5. The control unit 13 comprises an input means 14 like a button which can be pressed for allowing a user to initiate and/or stop a sterilization process via the input unit 14. The input unit 14 can also be adapted to allow the user to input desired sterilization parameters like temperature and duration. The input means 14 can also be a keyboard or a computer mouse which allows a user in cooperation with a graphical user interface to set desired sterilization parameters.

The sterilization coat 1 and the control unit 13 can be regarded as a sterilization apparatus for sterilizing a device 2.

Also the sterilization coat 101 described above with reference to FIG. 3 is connected to a control unit comprising a voltage source, wherein the control unit comprises an input means allowing a user to control the sterilization process performed by the sterilization coat 101.

In the following a sterilization method for sterilizing a device will be exemplarily described with reference to a flowchart shown in FIG. 4.

In step 201, the sterilization coat is arranged on a detection device.

In step 202, a user can initiate a sterilization procedure and preferentially input desired sterilization parameters.

In step 203, the sterilization coat heats for sterilizing the device. Preferentially, a control unit controls a heating layer of the sterilization coat such that the heating layer heats to a temperature being sufficient for sterilization purposes, for example, the heating layer heats to a temperature above 140° C. The heat generated by the heating layer can pass the outer electrically isolating and thermally conductive layer such that the outer surface of the sterilization coat is heated. The electrically isolating and thermally isolating layer arranged between the heating layer and the device ensures that the device is not adversely affected by the sterilization procedure.

In step 204, the time of the performed sterilization procedure is indicated by an indicator of the sterilization code.

If the sterilization coat is already arranged on the device, step 201 can be omitted. Moreover, in an embodiment also step 204 can be omitted.

The device is preferentially a device to be used in a medical procedure like a flat panel X-ray detector. However, the device can also be another device which has to be sterilized, in particular, also in a non-medical procedure like in procedures for treating food.

The control unit is preferentially adapted to start and stop the sterilization process automatically and/or by a user via input means. The indicator preferentially visibly shows if and/or when the sterilization process took place.

The sterilization coat preferentially provides simplicity in hospital sterilization, especially in any place in the medical arena where one wishes to sterilize sizable areas. For example, the detection device is preferentially an X-ray detector augmented with several optical cameras for patient tracking. The sterilization coat can be adapted to be applied to such a detection device for sterilizing such a detection device, wherein the detection of the X-rays and the imaging by the optical cameras is substantially not adversely affected by the sterilization coat.

The heating of the heating layer is preferentially a standard ohmic heating. Due to the non-zero resistance of the heating layer having preferentially a resistance between 10 and 50 Ohm an electrical current that runs through the heating layer will result in an increase of the temperature of the heating layer. The heating layer has preferentially a constant square resistance over its surface such that the amount of energy dissipated will be substantially the same everywhere. As a result, the amount of heating is also substantially the same for all locations on the surface of the heating layer. This reduces the probability that parts will be over heated or under heated.

The thermal expansion coefficients of the heating layer and of the outer electrically isolating layer that is thermally conductive and/or of the thermally isolating layer that is preferentially also electrically isolating between the heating layer and the device are preferentially matched for prolonging the lifetime of the sterilization coat.

It is further preferred that the thermally isolating layer between the heating layer and the device, and the heating layer are clamped to each other. This reduces the thermal conductivity of the heat generated in the heating layer towards the thermally isolating layer and thus further decreases the probability that the device is adversely affected by the heat generated in the heating layer. Although in the above described embodiments the sterilization coat comprises three layers, i.e. a heating layer, an electrically isolating layer that is thermally conductive and a thermally isolating layer between the device and the heating layer, the sterilization coat can also comprise more than these three layers. In particular, the thermally isolating layer does not have to be arranged directly on the heating layer and/or directly on the device. It is just preferred that the thermally isolating layer is arranged between the heating layer and the device, wherein also further layers can be arranged between the heating layer and the thermally isolating layer and/or between the thermally isolating layer and the device. Moreover, also the electrically isolating layer that is thermally conducting does not have to be arranged directly on the heating layer and does not have to form an outer surface of the sterilization coat. Also a further layer can form the outer surface of the sterilization coat and a layer can be arranged between the heating layer and the electrically isolating layer that is thermally conductive.

Although in the above described embodiments, the electrically isolating layer that is thermally conductive, the heating layer and the thermally isolating layer between the heating layer and the device have been described as comprising certain materials, in other embodiments these layers can comprise other materials as long as these other materials ensure that the heating layer is heatable, the thermally isolating layer is thermally isolating and the electrically isolating layer that is thermally conductive is indeed electrically isolating and thermally conductive.

Although in the above described embodiments, the sterilization coat comprises an electrically isolating layer that is thermally conductive for safety reasons, in other embodiments this layer can be omitted.

Although in FIG. 1 the control unit is shown as a part being outside of the device, this unit can also be part of the device, for example, it can be included within the device.

Although in the above described embodiments the control unit comprises a voltage source for electrical heating, in other embodiments other control units can be used. For example, the control unit can comprise a light source for allowing the heating layer to generate heat optically or the control unit can comprise a magnetic source for allowing the heating layer to inductively generate the heat.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Any reference signs in the claims should not be construed as limiting the scope.

The invention relates to a sterilization coat for sterilizing a device like a medical detection device, wherein the sterilization coat is adapted to be arranged on the device and comprises a heating layer for sterilizing the device by heating. If the sterilization coat has been arranged on the device, several sterilization procedures can be performed by heating the sterilization coat using the heating layer. Thus, it is not necessary to arrange sterile plastic drapes around the device each time a sterile device is needed, for example, before each medical interventional procedure. If a sterilization of the device is needed, the heating layer heats the device for sterilizing. This simplifies the procedure for sterilizing the device.

1. A sterilization coat for sterilizing a device, wherein the sterilization coat (1;
101) is adapted to be arranged on the device (2) and comprises a heating layer (5) for sterilizing the device (2) by heating.

2. The sterilization coat as defined in claim 1, wherein the sterilization coat (1; 101) is adapted to be arranged on a detection device (2) for detecting radiation, wherein the sterilization coat (1; 101) is transparent to the radiation.

3. The sterilization coat as defined in claim 1, wherein the heating layer (5) has a first side (7) and a second side (8) being opposite to the first side, wherein the sterilization coat (1; 101) comprises a thermally isolating layer (6) arranged at the first side.

4. The sterilization coat as defined in claim 3, wherein the thermally isolating layer (6) is electrically isolating.

5. The sterilization coat as defined in claim 1, wherein the heating layer (5) has a first side (7) and a second side (8) being opposite to the first side, wherein the sterilization coat (1; 101) comprises an electrically isolating layer (4) that is thermally conductive and arranged at the second side.

6. The sterilization coat as defined in claim 1, wherein the sterilization coat (101) comprises a first region (108) being transparent to a first kind of radiation and a second region (109) being transparent to a second kind of radiation.

7. The sterilization coat as defined in claim 6, wherein the heating layer comprises a first heating material being transparent to the first kind of radiation in the first region (108) and a second heating material being transparent to the second kind of radiation in the second region (109), wherein the first heating material and the second heating material are electrically connected to each other.

8. The sterilization coat as defined in claim 1, wherein the sterilization coat (1; 101) comprises an indicator (111) for indicating if the heating layer has sterilized the device (2).

9. A sterilization apparatus for sterilizing a device by heating, the sterilization apparatus comprising:
a sterilization coat (1; 101) as defined in claim 1,
a control unit (13) for controlling the heating by the heating layer (5).

10. The sterilization apparatus as defined in claim 9, wherein the control unit (13) and the heating layer (5) are adapted to generate heat electrically for sterilization.

11. A device comprising the sterilization coat as defined in claim 1 for sterilizing the device (2) by heating.

12. The device as defined in claim 11, wherein the device (2) is a detection device (2) for detecting radiation.

13. The device as defined in claim 12, wherein the detection device (2) comprises a first detector (106) for detecting a first kind of radiation and a second detector (107) for detecting a second kind of radiation.

14. The device as defined in claim 13, wherein the sterilization coat (101) comprises a first region (108) being transparent to the first kind of radiation and a second region (109) being transparent to the second kind of radiation, wherein the first region (108) is arranged on the first detector (106) and the second region (109) is arranged on the second detector (107).

15. A sterilization method for sterilizing a device, wherein a sterilization coat (1; 101) comprising a heating layer (5) is arranged on the device (2) and wherein the heating layer (5) heats for sterilizing the device (2).
